(19) United States
(12) Patent Application Publication
(10) Pub. No.: US 2008/0154090 A1
(43) Pub. Date: Jun. 26, 2008

(54) Endoscopic System for In-Vivo Procedures

(75) Inventor: Dan Hashimshony, Givat Ada (IL)

Correspondence Address: Martin D Moynihan;PRTSI INC, P o box 16446, Arlington, VA, 22215, US

(73) Assignee: Dune Medical Devices Ltd., Caesaria (IL)

(21) Appl. No.: 10/567,581

(22) PCT Filed: Jan. 4, 2006
(86) PCT No.: PCT/IL2006/000015

§ 371 Date: Feb. 8, 2006

Related U.S. Application Data
(60) Provisional application No. 60/641,081, filed on Jan. 4, 2005.
(60) Provisional application No. 60/665,842, filed on Mar. 29, 2005.

(51) Int. Cl. A61B 1/018; A61M 25/09

(52) U.S. Cl. 600/104; 600/101; 600/585

(57) ABSTRACT

An endoscopic system for in-vivo tissue characterization, using a nonirradiative electromagnetic sensor, is described. The endoscopic system is further configured to employ several follow-up procedures, for example, biopsy sampling, localized surgery, dispensing a medicament, and the like, so that on the whole, the endoscopic system provides for the early detection of cancerous and pre-cancerous tissue, in vivo, and for the application of immediate follow-up procedures to any such tissue.

### FIELD AND BACKGROUND OF THE INVENTION

The present invention relates to an endoscopic system for in-vivo tissue characterization, employing a nonirradiative electromagnetic sensor.

The impact of cancer is great. In spite of enormous expenditures of financial and human resources, early detection of malignant tumors remains an unfulfilled medical goal. While it is known that a number of cancers are treatable if detected at an early stage, lack of reliable screening procedures results in their being undetected and untreated.

Various forms of endoscopes are currently in use. For example, diagnosis of different conditions of the colon generally involves using a colonoscope. A typical colonoscope includes, at its distal end, with respect to an operator, a light source, a video chip, and a suction channel. These elements are all in communication with a proximal end of the colonoscope via wires and channels housed within a flexible tube. The distal end is inserted into a patient's rectum and can be maneuvered along the length of the colon. A colonoscope can be inserted far enough into a patient's colon for the distal end to enter the patient's cecum. The tip of the colonoscope can also be maneuvered through the ileo-cecal valve into the terminal ileum.

A colonoscope provides a visual image only of the region of the colon that is immediately near the light source and video chip, yielding visual information for only a small region of the colon at any given time. Lesions in a patient's colon typically are identified by progressive and painstaking visual examination of the entire colon. However, a single colonoscopy is often not sufficient to identify the source of colorectal bleeding which is typically sporadic and in many cases would be best located by observing the entire colon over a period of time.

Various attachments to a colonoscope allow small surgical procedures, such as tissue biopsies, to be carried out during a colonoscopic examination.

Endoscopy of the small intestine is also known. For example, U.S. Pat. No. 5,984,860, to Shan, entitled, “Pass-through duodenal enteroscopic device,” whose disclosure is incorporated herein by reference, describes a tethered ingestible, enteroscopic video camera, which utilizes the natural contraction wave of the small intestine to propel it through the small intestine at about the same speed as any other object therein. The video camera includes an illumination source at its forward end. Covering the camera lens and illumination source is a transparent inflatable balloon, adapted to gently expand the small intestine immediately forward the camera for better viewing. A small diameter communication and power cable unwinds through an aperture in the rear of the camera as it moves through the small intestine. Upon completion of movement through the small intestine the cable is automatically separated, permitting the cable to be withdrawn through the stomach and intestine. The camera continues through the large intestine and passes from the patient through the rectum.

The aforementioned endoscopes, while providing means to access and visualize portions of the gastrointestinal track, do not provide means of detecting gastrointestinal pathologies, which are not clearly visible. In particular, they do not provide means for localization and differentiation of occult tumors. Typically, a large tumor is readily located by visualization. Yet, for subsequent operative success, as well as for the success of other forms of treatment, it is necessary to somehow locate tumors in their occult stage, when they cannot be found by sight and feel.

Similarly, lung cancer is the leading cause of cancer death in both men and women in Western society. When detected and treated at an early stage, before it has spread to lymph nodes or other organs, the five-year survival rate is about 42%. However, detection at an early stage is rare. The five-year survival rate for all stages of lung cancer combined is about 14%—a factor of three lower.

Most patients are diagnosed when exhibiting symptoms, for example by bronchoscopy, using an endoscope specifically designed for the lungs. The walls of the bronchial tubes are examined, for example, visually, and small pieces of tissue may be removed for biopsy. Alternatively, needle aspiration biopsy may be performed, by inserting a needle between the ribs to draw cells from the lung. Alternatively, surgery is performed to remove tissue for biopsy. Diagnosis for malignancy is generally made in a laboratory, on the removed biopsy sample, by examination of the characteristics of the cells under a microscope.

However, biopsy diagnosis performed in a laboratory and follow up procedures based on laboratory biopsy suffer from inherent disadvantages, as follows:

i. biopsy is generally performed when symptoms are observed, and the cancer is at an advanced stage;

ii. it may happen that the biopsy is taken from a region near the tumor, and not the tumor itself, leading to erroneous false negative results;

iii. the exact location from which the biopsy was taken, may be difficult to reproduce; and

iv. The results of the biopsy examination are not immediate.

Thus, devices and methods for the early detection of cancerous and pre-cancerous tissue, in vivo, are highly desirable.

### SUMMARY OF THE INVENTION

The present invention successfully addresses the shortcomings of the presently known configurations by providing an endoscopic system for in-vivo tissue characterization, using a nonirradiative electromagnetic sensor. The endoscopic system is further configured to employ several follow-up procedures, for example, biopsy sampling, localized surgery, dispensing a medicament, and the like, so that on the whole, the endoscopic system provides for the early detection of cancerous and pre-cancerous tissue, in vivo, and for the application of immediate follow-up procedures to any such tissue.

In accordance with one aspect of the present invention, there is thus provided an endoscope, which comprises:

an intracorporeal portions, configured for insertion into a body, and including:

a nonirradiative electromagnetic sensor for tissue characterization;

a communication line, on which the nonirradiative electromagnetic sensor is mounted; and

an extracorporeal portion.

Additionally, the communication line is formed as an instrument bundle.

Furthermore, the instrument bundle extends beyond a distal-most end of the endoscope, with respect to an operator, and a distal-most end of the instrument bundle may be manipulated, extracorporeally, to bring the nonirradiative electromagnetic sensor to contact with a tissue, for characterization.

Additionally, the intracorporeal portion further includes an instrument channel, and wherein the nonirradiative electromagnetic sensor for tissue characterization is inserted into the instrument channel.

Furthermore, the nonirradiative electromagnetic sensor for tissue characterization may be removed from the instrument channel and replaced with another instrument.

Additionally, the endoscope may further include a catheter, wherein the nonirradiative electromagnetic sensor is inserted into the catheter, and the catheter is inserted into the instrument channel.

Furthermore, the catheter may extend beyond a distal-most end of the endoscope, with respect to an operator, and a distal-most end of the catheter may be manipulated independently of the distal-most end of the endoscope.

Additionally, the intracorporeal portion further includes an optical channel for an optical instrument.

Furthermore, the optical instrument is configured to observe the nonirradiative electromagnetic sensor.

Additionally or alternatively, the intracorporeal portion further includes a second instrument.

Furthermore, the second instrument is selected from the group consisting of an optical sensor, an X-ray sensor, an RF sensor, a MW sensor, an infrared thermography sensor, or an ultrasound sensor, an MR sensor, an impedance sensor, a temperature sensor, a biosensor, a chemical sensor, a radioactive-emission sensor, and a mechanical sensor.

Additionally, the second instrument is configured to sense the nonirradiative electromagnetic sensor.

Furthermore, the intracorporeal portion is designed for motion in a body lumen.

Additionally, the intracorporeal portion is designed for reaching the lumen by percutaneous insertion.

Furthermore, the endoscope is configured for characterizing a tissue along the lumen wall.

Alternatively, the endoscope is configured for characterizing a tissue outside the lumen, by penetrating the lumen wall.

Additionally, the body lumen is selected from the group consisting of an oral cavity, a nostril, an esophagus, a gastrointestinal tract, a rectum, a colon, bronchi, a vagina, a cervix, a urinary tract, a bladder, a uterus, and blood vessels.

Alternatively, the intracorporeal portion is designed for insertion through a trocar valve.

Additionally, tissue characterization relates to the detection of a malignancy.

Additionally or alternatively, tissue characterization relates to the detection of a pre-cancerous state.

In accordance with another aspect of the present invention, there is thus provided a method of tissue characterization, which comprises:

inserting a nonirradiative electromagnetic sensor intracorporeally; and

characterizing an intracoroporeal tissue.

In accordance with still another aspect of the present invention, there is thus provided an in-vivo method, comprising:

providing an endoscope, having an instrument channel;

inserting a sensor for tissue characterization, mounted on communication line, into the instrument channel;

characterizing a tissue;

removing the sensor for tissue characterization;

inserting a second instrument into the instrument channel, to the location of the characterized tissue; and

performing a second procedure with the second instrument.

In accordance with yet another aspect of the present invention, there is thus provided an in-vivo method, comprising:

providing an endoscope, having an instrument channel;

inserting a sensor for tissue characterization, mounted on a communication line, into the instrument channel;

extending the sensor, mounted on the communication line, to beyond the reach of the instrument channel;

characterizing a tissue;

inserting a guide wire to the location of the characterized tissue;

removing the sensor for tissue characterization;

inserting a second instrument into the instrument channel, along the guide wire, to the location of the characterized tissue; and

performing a second procedure with the second instrument.

In accordance with still another aspect of the present invention, there is thus provided a method for tissue characterization, comprising:

inserting a guide wire intracorporeally;

inserting a sensor for tissue characterization, mounted on a communication line, intracorporeally, along the guide wire; and

characterizing the tissue with the sensor.

In accordance with still another aspect of the present invention, there is thus provided an endoscope system, which comprises:

an intracorporeal portions, configured for insertion into a body, and including:
a nonirradiative electromagnetic sensor for tissue characterization;
a communication line, on which the nonirradiative electromagnetic sensor is mounted; and

an extracorporeal portion;

a control unit; and

a signal analyzer.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A and 1B schematically illustrate an overall endoscopic system, in accordance with embodiments of the present invention;

FIG. 2 schematically illustrates an intracorporeal portion of an endoscope, in accordance with embodiments of the present invention;

FIGS. 3A-3C schematically illustrate an intracorporeal distal tip of an endoscope, and the synergy between a sensor and an optical instrument at the distal tip, in accordance with embodiments of the present invention;

FIGS. 3D-3H schematically illustrate different embodiments of an intracorporeal portion of an endoscope of the present invention;

FIGS. 4A-4D further illustrate an endoscopic system, in accordance with embodiments of the present invention;

FIGS. 5A-5D summarize different manners of motion in the body, in accordance with embodiments of the present invention;

FIGS. 6A-6D schematically illustrate tissue characterization coupled with at least one additional procedure, in accordance with embodiments of the present invention;

FIGS. 7A and 7B schematically illustrate tissue characterization coupled with at least one additional procedure, in accordance with other embodiments of the present invention; and

FIGS. 8A-8C schematically illustrate sensor insertion along a guide wire, in accordance with embodiments of the present invention.

### DESCRIPTION OF THE PREFERRED EMBODIMENTS

The present invention relates to an endoscopic system for in-vivo tissue characterization, using a nonirradiative electromagnetic sensor. The endoscopic system is further configured to employ several follow-up procedures, for example, biopsy sampling, localized surgery, dispensing a medicament, and the like, so that on the whole, the endoscopic system provides for the early detection of cancerous and pre-cancerous tissue, in vivo, and for the application of immediate follow-up procedures to any such tissue.

The principles and operation of the device and method according to embodiments of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

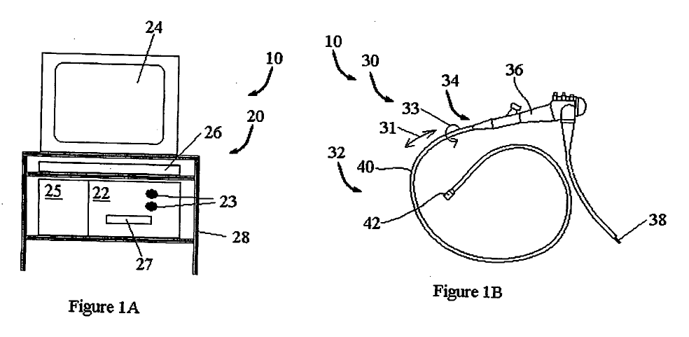

Referring now to the drawings, FIGS. 1A and 1B illustrate an overall endoscopic system 10, in accordance with embodiments of the present invention.

The endoscopic system 10 preferably includes an extracorporeal control station 20, having a control unit 22, preferably, having control buttons 23, and possibly also, an input interface, such as a keyboard 26, and a read/write device 27. The control unit 22 is in communication with a signal analyzer 25, and possibly, with a display screen 24.

The control station 20 may be placed on a rack 28. Alternatively, a hand-held device, or a laptop, as known, may be used.

Additionally, the endoscopic system 10 includes an endoscope 30, having an extracorporeal portion 34, which preferably includes a manipulator 36, for manipulating the endoscope 30, and a connector 38, for connecting to the extracorporeal control station 20.

Furthermore, the endoscope 30 includes an intracorporeal portion 32, designed for insertion into a body, for example, into a lumen or a trocar valve, and formed as a flexible tubing 40, having a distal tip 42, with respect to an operator (not shown).

The manipulator 36 is preferably, handheld. It may include both mechanical and electrical control features, for controlling the position of the tubing 40 and its tip 42. Preferably, the manipulator 36 may apply to the flexible tubing 40 both lateral motion, as seen by the arrow 31, and rotational motion, as seen by the arrow 33.

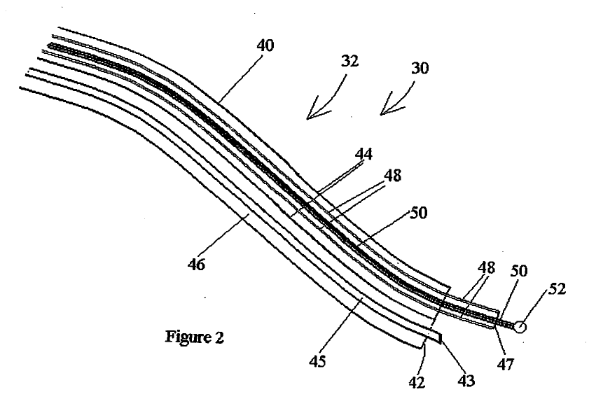

Referring further to the drawings, FIG. 2 schematically illustrates the intracorporeal portion 32 of the endoscope 30, in accordance with embodiments of the present invention.

Preferably, the flexible tubing 40 of the intracorporeal portion 32 includes an instrument channel 44. Additionally, a sensor 52 is configured for insertion into the instrument channel 44, preferably, within a catheter 48. The sensor 52 is mounted on a communication line 50 for signal transmission, which is preferably formed as an instrument bundle 50. The instrument bundle 50 may include a power cable, a communication line for signal transmission, data cables, and a mechanical control cable.

The sensor 52 may be a nonirradiative electromagnetic sensor for tissue characterization, for example, as taught in commonly owned U.S. Pat. No. 6,813,515, to Hashimshony, whose disclosure is incorporated herein by reference. U.S. Pat. No. 6,813,515 describes a nonirradiative electromagnetic sensor, which applies an electrical pulse to a tissue, thus generating an electrical fringe field in the zone of the tissue and producing a reflected pulse therefrom with negligible radiation penetrating into the tissue itself. The sensor detects the reflected electrical pulse and compares the electrical characteristics of the reflected electrical pulse with respect to the applied electrical pulse to provide an indication of the dielectric properties of the examined tissue.

Alternatively, the sensor 52 may be a nonirradiative electromagnetic sensor for tissue characterization, as taught in commonly owned U.S. Patent Application 60/665,842, whose disclosure is incorporated herein by reference. U.S. Patent Application 60/665,842 describes a sensor for tissue characterization, comprising: a resonating element, formed as a conductive structure, configured to be placed proximally to an edge of a tissue for characterization, without penetrating the tissue, and having a diameter-equivalent D, which defines a cross-sectional area of the resonating element, on a plane substantially parallel with the edge; and at least one conductive lead, for providing communication with an external system, wherein the resonating element is configured to resonate at a free-air wavelength range of between about λ and about 10λ, wherein λ is at least about ten times the diameter-equivalent D, and wherein upon receiving a signal in the range of between about λ and about 10λ, the sensor is configured to induce electric and magnetic fields, in a near zone, in the tissue, the near zone being a hemisphere having a diameter of substantially D, beginning with the edge, while causing negligible radiation in a far zone, so that the tissue, in the near zone, effectively functions as part of the resonating element, varying a resonating response to the sensor, and so the tissue, in the near zone, is thereby characterized by its electromagnetic properties, by the resonating response to the sensor.

It will be appreciated that in accordance with embodiments of the present invention, other electromagnetic sensors may be used.

It will be appreciated that generally, the flexible tubing 40 also includes an optical channel 46, for an optical instrument 43, mounted on an optical communication line 45, preferably formed as an optical fiber 45. Alternatively, an optical bundle 45 may be used, including, for example, a power cable, optical data cables, and a mechanical control cable.

Preferably, tissue characterization is performed both visually, by the optical instrument 43, and via the sensor 52. However, the present invention may be operable also without the optical channel 46 and without the optical instrument 43.

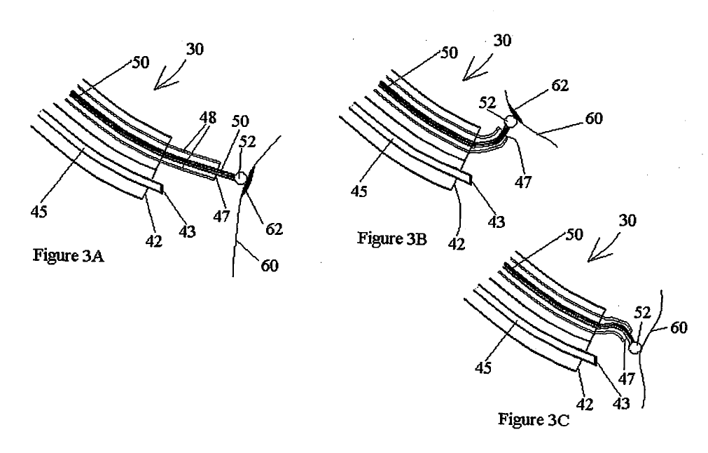

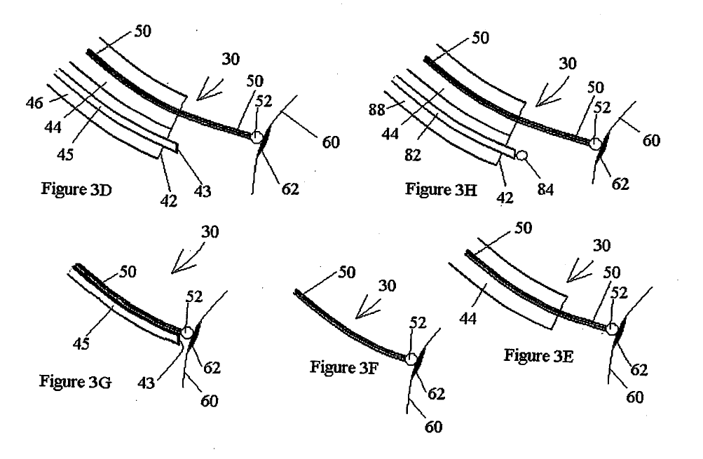

Referring further to the drawings, FIGS. 3A-3C schematically illustrate the intracorporeal distal tip 42 of the endoscope 30, and the synergy between the sensor 52 and the optical instrument 43, in accordance with embodiments of the present invention.

Preferably, the catheter 48 has a distal tip 47, which may extend beyond the distal tip 42 of the endoscope. Additionally, the catheter 48 may be manipulated, independent of the tubing 40, via the instrument bundle 50, as seen in FIGS. 3A-3C, so that the sensor 52 may be brought in contact with a specific location of a tissue 60, such as the inner wall of a body lumen or another tissue location, for characterizing a suspected anomaly 62, as seen in FIGS. 3A and 3B. The manipulation of the catheter 48 may be mechanical, for example, via wires, or electronic, as known.

Additionally, the sensor 52 may be brought in contact with a healthy portion of the tissue 60, as seen in FIG. 3C, for characterization of a reference tissue.

Alternatively, the catheter 48 is not used, yet the instrument bundle 50 may extend beyond the distal tip 42 of the endoscope, and a distal-most end of the instrument bundle 50 may be manipulated, extracorporeally, to bring the sensor 52 to contact with the tissue 60, for characterization.

Referring further to the drawings, FIGS. 3D-3H schematically illustrate different embodiments of the intracorporeal portion 32 of the endoscope 30 of the present invention.

FIG. 3D describes another embodiment, wherein no catheter 48 is used, and the sensor 52, mounted on the instrument bundle 50, is inserted directly into the instrument channel 44.

FIG. 3E describes still another embodiment, wherein the flexible tubing 40 has a single lumen, forming the instrument channel 44. No optical channel 46 is used.

FIG. 3F describes yet another embodiment, wherein the instrument bundle 50 is integrated with the flexible tubing 40.

FIG. 3G describes still another embodiment, wherein the instrument bundle 50 and the optical bundle 45 form the flexible tubing 40.

FIG. 3H describes yet another embodiment, wherein the intracorporeal portion 32 has two channels, the instrument channel 44 in which the sensor 52 moves, mounted on the instrument bundle 50, and a second channel 88, into which a second instrument 84 may be inserted, mounted on a second instrument bundle 82.

The second sensor 84 may be any one of an optical sensor, an x-ray sensor, an RF sensor, a MW sensor, an infrared thermography sensor, an ultrasound sensor, an MR sensor, an impedance sensor, a temperature sensor, a biosensor, a chemical sensor, a radioactive-emission sensor, a mechanical sensor, and (or) another tissue characterization sensor, as known.

Preferably, the sensor 52 is visible on the second modality of the second sensor 84.

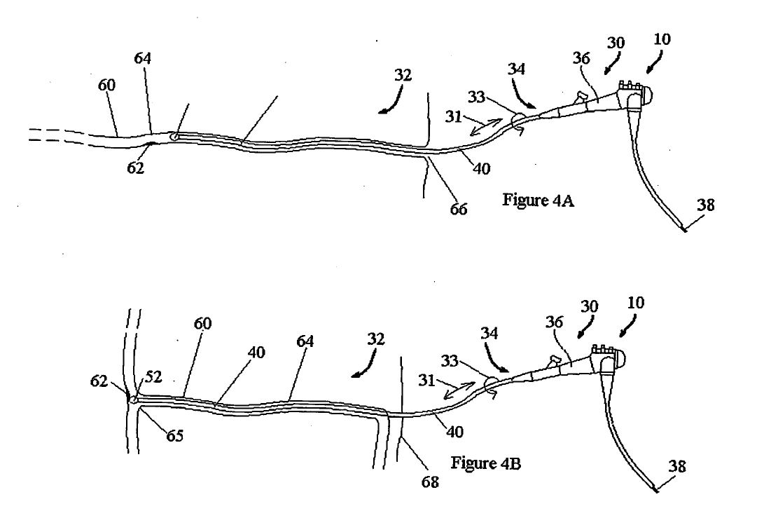

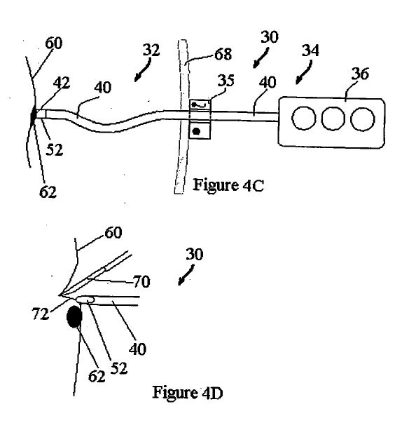

Referring further to the drawings, FIGS. 4A-4D further illustrate the intracorporeal portion 32 of the endoscope 30, in accordance with embodiments of the present invention.

As seen in FIG. 4A, the endoscope 30 may be inserted in a body lumen 64, for characterizing the tissue 60 formed as the walls of the body lumen 64. The insertion may be via a body opening 66, such as a mouth, a nose, or another body opening or orifice.

As seen in FIG. 4B, the endoscope 30 may be inserted percutaneously, through a skin 68, and then into the body lumen 64, for characterizing the tissue 60 formed as the walls of the body lumen 64, for example, when the body lumen 64 is a blood vessel.

Additionally, as seen in FIG. 4B, the tissue which is characterized may be at a lumen junction 65.

As seen in FIGS. 4C and 4D, the endoscope 30 may be inserted via a trocar valve 35, through the skin 68, for characterizing the tissue 60, for example, during a minimally invasive surgery.

In accordance with embodiments of the present invention, the tissue 60, which is characterized by the sensor 52 may be the walls and (or) junctions of the body lumen 64, the walls of other body cavities which may be reached by body lumens, for example, the stomach or the uterus, or open flesh, during a minimally invasive surgery. Additionally, tissue characterization may include penetrating the lumen and characterizing the tissue bulk.

In accordance with one embodiment, the sensor 52 may be guided along the body lumen 64, characterizing the tissue 60, substantially along the full length of it.

Alternatively, the sensor 52 may be guided along the body lumen 64, characterizing the tissue 60, along predetermined portions of it.

Additionally or alternatively, it may happen that the optical instrument 43 detects the suspected anomaly 62, visually, and the sensor 52 is manipulated so as to be brought in contact with the suspected anomaly 62 and characterize it.

Additionally or alternatively, other imaging modalities, such as x-ray, MRI, ultrasound, or another non-invasive modality, detects the suspected anomaly 62, and the sensor 52 is manipulated so as to be brought in contact with it and characterize it.

Alternatively, as seen in FIGS. 4C and 4D, during a minimally invasive surgery, the sensor 52 may be used in two manners, as follows:
i. for characterizing the tissue 60 and identifying the anomaly 62; and
ii. during the removal of the anomaly 62, by a surgical instrument 70, characterizing a wall of a cut 72, to ensure that it is formed of a healthy tissue, and that the anomaly 62 is contained within.

It will be appreciated that the endoscope 30 may be a multi-channel endoscope, so that several instruments, for example, the optical instrument 43, the sensor 52, and another instrument; for example, the surgical instrument 70 may operate together. Alternatively, only one or two channels may be available, and instruments are pulled out and replaced with other instruments, as needed.

Preferably, the sensor 52 is visible on other imaging modalities such as x-rays, ultrasound and MRI, and may be guided using another imaging modality, so that it can be guided to zones which are not accessible to the optical instrument 43 or in cases where the optical instrument 43 is not used.

Preferably, the catheter 48 is between about 0.5 and 4 mm in diameter, the sensor 52 is between about 0.3 and 3 mm in diameter, the instrument bundle is about 2 mm in diameter, and the intracorporeal portion 32 is between about 2 and 5 mm. It will be appreciated that other dimensions, which may be larger or smaller, may similarly be used.

The measurement is preferably performed by reflection of electromagnetic fields from the near vicinity of the sensor 52, for example, as taught in commonly owned U.S. Pat. No. 6,813,515, to Hashimshony, whose disclosure is incorporated herein by reference. Alternatively, the measurement is performed as taught in commonly owned U.S. Patent Application 60/665,842, whose disclosure is incorporated herein by reference. It will be appreciated that in accordance with embodiments of the present invention, other electromagnetic sensors may also be used.

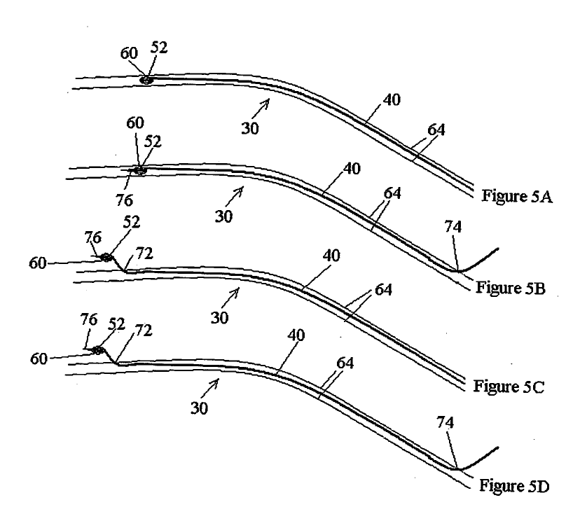

Preferably, the control unit 22 of the extracorporeal control station 20 analyzes the reflection and displays results. It will be appreciated that another computer may be used, as known. The results may be used for characterization of the tissue 60, such as the lumen wall 60, for example, the broncos wall 60, and the anomaly 62. It will be appreciated that the tissue 60 may be a portion of tissue which is not part of a lumen wall, for example, as illustrated in FIGS. 5C and 5D, hereinbelow. The results may be produced graphically, numerically, or as positive or negative answers. The results may also be presented textually.

The results may be relative, that is, a comparison between the anomaly 62 of different types and the reference tissue 60, or several references of the tissue 60 taken from different locations. Alternatively, the results may be based on literary data, in which the tissue is characterized based on previous tests and (or) data found in the literature.

The tissue characterization relating to the anomaly 62 may relate to the detection of a malignancy, or a pre-cancerous state. Additionally or alternatively it may relate to the detection of another pathology, for example, internal bleeding.

Referring further to the drawings, FIGS. 5A-5D summarize the different manners of the endoscopic system's motion in the body, in accordance with embodiments of the present invention.

As seen in FIG. 5A the flexible tubing 40 of the endoscope 30 moves entirely within a body lumen 64, for characterizing the tissue 60 along the lumen wall. The entry point is a bodily orifice, such as the oral cavity, a nostril, the rectum, the vagina, the urinary orifice or another bodily orifice.

As seen in FIG. 5B the flexible tubing 40 of the endoscope 30 moves within the body lumen 64, but entry is percutaneous, at an entry point 74. Preferably, the sensor 52 is associated with a sharp edge 76, to facilitate the entry. For example, the lumen may be a blood vessel, and the entry point may be a femoral vain or a jugular vain. It will be appreciated that other points of percutaneous entry are similarly possible.

As seen in FIG. 5C, the entry point is a bodily orifice, but for characterizing the tissue 60, beyond the lumen 64, the sensor 52 penetrates the lumen 64 at a point 72. Preferably, the sensor 52 moves within the lumen to a point as near as possible to the site for measurement, then penetrates the lumen. Preferably, the sensor 52 is associated with the sharp edge 76, to facilitate the penetration.

As seen in FIG. 5D, the sensor 52 enters the lumen percutaneously, at the entry point 74 and penetrates the lumen 64 at a point 72, for characterizing the tissue 60 beyond the lumen 64.

As has been pointed out, biopsy diagnosis performed in a laboratory and follow up procedures based on laboratory biopsy suffer from inherent disadvantages, as follows:

i. biopsy is generally performed when symptoms are observed, and the cancer is at an advanced stage;

ii. it may happen that the biopsy is taken from a region near the tumor, and not the tumor itself, leading to erroneous false negative results;

iii. the exact location from which the biopsy was taken, may be difficult to reproduce; and

iv. the results of the biopsy examination are not immediate.

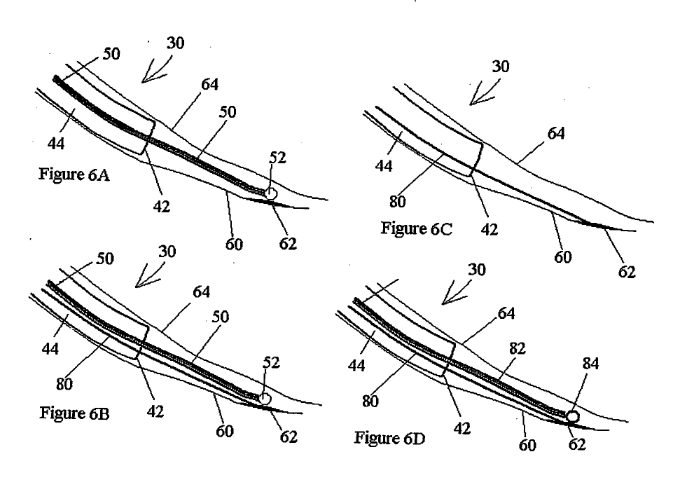

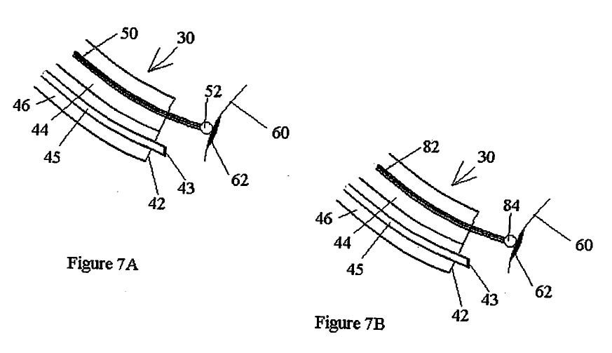

The present invention seeks to provide for the application of immediate follow-up procedures directly with the detection of cancerous and pre-cancerous tissue, in vivo. Thus, methods are provided for the insertion of additional instruments to the characterized site, upon a detection of an anomaly. These instruments may be directed at additional characterization by other sensors, biopsy sampling, performing localized surgery, dispensing medication, and (or) other procedures. These methods are described hereinbelow, in conjunction with FIGS. 6A-6D and 7A-7B.

Referring further to the drawings, FIGS. 6A-6D schematically illustrate another method of tissue characterization preferably coupled with at least one additional procedure, in accordance with embodiments of the present invention.

In some cases the reach of the endoscope is restricted by its diameter of about 2-3 mm, yet it is desired to reach beyond it, with the sensor 52, mounted on the instrument bundle 52, whose diameter may be as small as about 0.3 mm.

Thus, as seen in FIG. 6A, the sensor 52 extends beyond the distal tip 42 of the instrument channel 42 and characterizes an anomaly 62 of the tissue 60.

As seen in FIG. 6B, a guide wire 80 is inserted into the instrument channel 44, to the location of the sensor 52.

As seen in FIG. 6C, the sensor 52 is removed, after the characterization.

As seen in FIG. 6D, a second instrument 84, mounted on a second instrument bundle 82, is inserted into the instrument channel 44, to the location of the sensor 52, for performing at least one additional procedure on the tissue 60. The at least one additional procedure may be directed at additional characterization by another sensor, biopsy sampling, performing localized surgery, dispensing medication, and (or) another procedure.

It will be appreciated that the second instrument 84 may then be removed and another instrument still may be inserted in its place.

It will be appreciated that the second instrument 84 may be inserted without removing the sensor 52.

Referring further to the drawings, FIGS. 7A and 7B schematically illustrate performing a second procedure without a guide wire, in accordance with another embodiment of the present invention.

As seen in FIG. 7A, tissue characterization is performed by the sensor 52.

As seen in FIG. 7B, the sensor 52 is then removed, the second instrument 84 is inserted, mounted on the second instrument bundle 82, and a second procedure is performed at the characterized site, by the second instrument 84.

The second instrument 84 of FIGS. 6D and 7B may be a biopsy instrument, such as a biopsy brush, needle, or knife, an instrument for localized surgery, for example, by resection, ablation, for example, of ultrasound, RF, MW or another ablation method, or by cryosurgery, laser surgery, and the like, a dispensing instrument, for example, for dispensing a medication or for implanting brachytherapy seeds, or an instrument for other characterization and (or) treatment procedures.

It will be appreciated that the second instrument 84 may be a second sensor 84, for characterizing the tissue by a second modality. The second sensor 84 may be any one of an optical sensor, an x-ray sensor, an RF sensor, a MW sensor, an infrared thermography sensor, an ultrasound sensor, an MR sensor, an impedance sensor, a temperature sensor, a biosensor, a chemical sensor, a radioactive-emission sensor, a mechanical sensor, and (or) another tissue characterization sensor, as known.

Referring further to the drawings, FIGS. 5A-8C schematically illustrate sensor insertion along a guide wire, in accordance with embodiments of the present invention.

As seen in FIG. 5A, the guide wire 80 is inserted intracorporeally.

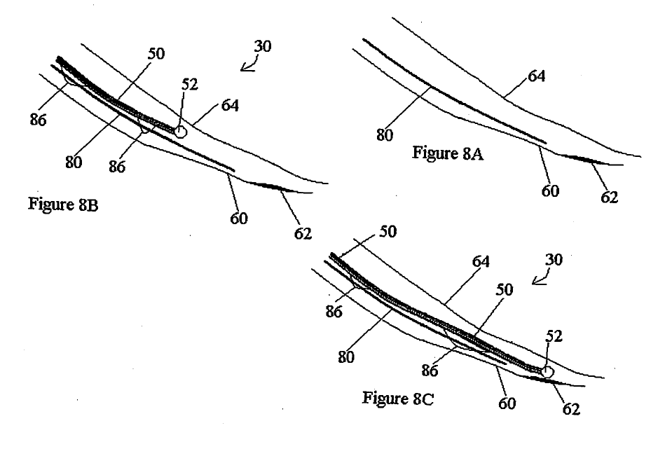

As seen in FIGS. 5B and 8C, the sensor 52, mounted on the instrument bundle 50 is wound on the guide wire 80 by wire loops 86 and is inserted along the guide wire 80 intracorporeally.

In accordance with embodiments of the present invention, the endoscope 30 may be designed for insertion in a body lumen, for example, an oral cavity, a nostril, an esophagus, a gastrointestinal tract, a rectum, a colon, bronchi, a vagina, a cervix, a urinary tract, a bladder, a uterus, and blood vessels, or another body lumen. Additionally or alternatively, it may be designed for insertion in a trocar valve.

It is expected that during the life of this patent many relevant devices and methods for tissue characterization in a body lumen, using an electromagnetic probe, mounted on an endoscopic device, may be developed and the scope of the present invention is intended to include all such new technologies a priori.

As used herein the term “about” refers to ±20%.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, any citation or identification of any reference in this application shall not be construed as an admission that such reference is available as prior art to the present invention.

1. An endoscope, which comprises:
an intracorporeal portions, configured for insertion into a body, and including:
an electromagnetic sensor for tissue characterizations, operative in an electromagnetic frequency range of less than 100 Ghz;
a communication line, on which the electromagnetic sensor is mounted; and
an extracorporeal portion, configured for manipulating the intracorporeal portion.

2. The endoscope of claim 1, wherein the communication line is formed as an instrument bundle.

3. The endoscope of claim 2, wherein the instrument bundle extends beyond a distal-most end of the endoscope, with respect to an operator, and a distal-most end of the instrument bundle may be manipulated, extracorporeally, to bring the electromagnetic sensor to contact with a tissue, for characterization.

4. The endoscope of claim 1, wherein the intracorporeal portion further includes an instrument channel, and wherein the electromagnetic sensor for tissue characterization is inserted into the instrument channel.

5. The endoscope of claim 4, wherein the electromagnetic sensor for tissue characterization may be removed from the instrument channel and replaced with another instrument.

6. The endoscope of claim 4, and further including a catheter, wherein the electromagnetic sensor is inserted into the catheter, and the catheter is inserted into the instrument channel.

7. The endoscope of claim 6, wherein the catheter extends beyond a distal-most end of the endoscope, with respect to an operator, and a distal-most end of the catheter may be manipulated independently of the distal-most end of the endoscope.

8. The endoscope of claim 1, wherein the intracorporeal portion further includes an optical channel for an optical instrument.

9. The endoscope of claim 1, wherein the optical instrument is configured to observe the electromagnetic sensor.

10. The endoscope of claim 1, wherein the intracorporeal portion further includes a second instrument.

11. The endoscope of claim 10, wherein the second instrument is selected from the group consisting of an optical sensor, an X-ray sensor, an RF sensor, a MW sensor, an infrared thermography sensor, or an ultrasound sensor, an MR sensor, an impedance sensor, a temperature sensor, a biosensor, a chemical sensor, a radioactive-emission sensor, and a mechanical sensor.

12. The endoscope of claim 10, wherein the second instrument is configured to sense the electromagnetic sensor.

13. The endoscope of claim 1, wherein the intracorporeal portion is designed for motion in a body lumen.

14. The endoscope of claim 13, wherein the intracorporeal portion is designed for reaching the lumen by percutaneous insertion.

15. The endoscope of claim 13, configured for characterizing a tissue along the lumen wall.

16. The endoscope of claim 13, configured for characterizing a tissue outside the lumen, by penetrating the lumen wall.

17. The endoscope of claim 13, wherein the body lumen is selected from the group consisting of an oral cavity, a nostril, an esophagus, a gastrointestinal tract, a rectum, a colon, bronchi, a vagina, a cervix, a urinary tract, a bladder, a uterus, and a blood vessel.

18. The endoscope of claim 1, wherein the intracorporeal portion is designed for insertion through a trocar valve.

19. The endoscope of claim 1, wherein tissue characterization relates to the detection of a malignancy.

20. The endoscope of claim 1, wherein tissue characterization relates to the detection of a pre-cancerous state.

21. A method of tissue characterization, which comprises:
providing an endoscope, comprising:
an intracorporeal portions, configured for insertion into a body, and including:
an electromagnetic sensor for tissue characterization, operative in an electromagnetic frequency range of less than 100 Ghz.
a communication line, on which the electromagnetic sensor is mounted; and
an extracorporeal portion, configured for manipulating the intracorporeal portion;
inserting the electromagnetic sensor intracorporeally; and
characterizing an intracoroporeal tissue.

22. The method of claim 21, wherein the electromagnetic sensor is mounted on an instrument bundle.

23. The method of claim 22, wherein the instrument bundle extends beyond a distal-most end of the endoscope, with respect to an operator, and further including manipulating a distal-most end of the instrument bundle, extracorporeally, to bring the electromagnetic sensor to contact with a tissue, for characterization.

24. The method of claim 21, wherein the electromagnetic sensor for tissue characterization moves within an instrument channel.

25. The method of claim 24, and further including:
after the characterizing the intracoroporeal tissue, removing the electromagnetic sensor for tissue characterization from the instrument channel;
inserting a second instrument to the instrument channel; and
performing a second procedure with the second instrument.

26. The method of claim 25, wherein the second procedure includes taking a biopsy sample.

27. The method of claim 25, wherein the second procedure includes a localized surgery.

28. The method of claim 25, wherein the second procedure includes dispensing medication.

29. The method of claim 25, wherein the second procedure includes characterizing the tissue by an additional sensor.

30. The method of claim 24, wherein the electromagnetic sensor for tissue characterization moves within a catheter, inserted into the instrument channel.

31. The method of claim 30, and further including manipulating a distal-most end of the catheter, extracorporeally, to bring the electromagnetic sensor to contact with a tissue, for characterization.

32. The method of claim 21, and further including inserting an optical instrument to visually observe the electromagnetic sensor as it makes contact with a tissue.

33. The method of claim 21, and further including inserting a second instrument for characterizing the tissue by a second modality, together with the electromagnetic sensor.

34. The method of claim 33, wherein the second instrument is selected from the group consisting of an optical sensor, an X-ray sensor, an RF sensor, a MW sensor, an infrared thermography sensor, or an ultrasound sensor, an MR sensor, an impedance sensor, a temperature sensor, a biosensor, a chemical sensor, a radioactive-emission sensor, and a mechanical sensor.

35. The method of claim 33, wherein the second instrument is configured to sense the electromagnetic sensor.

36. The method of claim 21, wherein the inserting includes:
inserting to a body lumen from a body orifice; and
characterizing a tissue along the body lumen.

37. The method of claim 21, wherein the inserting includes:
inserting to a body lumen from a body orifice;
penetrating the body lumen; and
characterizing a tissue beyond the body lumen.

38. The method of claim 21, wherein the inserting includes:
percutaneously inserting;
reaching a body lumen;
moving along the body lumen; and
characterizing a tissue along the body lumen.

39. The method of claim 21, wherein the inserting includes:
percutaneously inserting;
reaching a body lumen;
moving along the body lumen;
penetrating the body lumen; and
characterizing a tissue beyond the body lumen.

40. The method of claim 21, wherein the body lumen is selected from the group consisting of an oral cavity, a nostril, an esophagus, a gastrointestinal tract, a rectum, a colon, bronchi, a vagina, a cervix, a urinary tract, a bladder, a uterus, and a blood vessel.

41. The method of claim 21, wherein inserting includes inserting through a trocar valve.

42. The method of claim 21, wherein tissue characterization relates to the detection of a malignancy.

43. The method of claim 21, wherein tissue characterization relates to the detection of a pre-cancerous state.

44. An in-vivo method, comprising:
providing an endoscope, having an instrument channel;
inserting into the instrument channel an electromagnetic sensor for tissue characterization, operative in an electromagnetic frequency range of less than 100 Ghz and mounted on communication line;
characterizing a tissue;
removing the sensor for tissue characterization;
inserting a second instrument into the instrument channel, to the location of the characterized tissue; and
performing a second procedure with the second instrument.

45. The method of claim 44, wherein the electromagnetic sensor for tissue characterization is a nonirradiative electromagnetic sensor.

46. The method of claim 50, wherein the additional sensor is selected from the group consisting of an optical sensor, an x-ray sensor, an RF sensor, a MW sensor, an infrared thermography sensor, or an ultrasound sensor, an MR sensor, an impedance sensor, a temperature sensor, a biosensor, a chemical sensor, a radioactive-emission sensor, and a mechanical sensor.

47. The method of claim 44, wherein the second procedure includes taking a biopsy sample.

48. The method of claim 44, wherein the second procedure includes a localized surgery.

49. The method of claim 44, wherein the second procedure includes dispensing medication.

50. The method of claim 44, wherein the second procedure includes characterizing the tissue with an additional sensor.

51. An in-vivo method, comprising:
providing an endoscope, having an instrument channel;
inserting into the instrument channel an electromagnetic, sensor for tissue characterization, operative in an electromagnetic frequency range of less than 100 Ghz and mounted on a communication line;
extending the sensor, mounted on the communication line, to beyond the reach of the instrument channel;
characterizing a tissue;
inserting a guide wire to the location of the characterized tissue;
removing the sensor for tissue characterization;
inserting a second instrument into the instrument channel, along the guide wire, to the location of the characterized tissue; and
performing a second procedure with the second instrument.

52. The method of claim 51, wherein the electromagnetic sensor for tissue characterization is a nonirradiative electromagnetic sensor.

53. The method of claim 53, wherein the additional sensor is selected from the group consisting of an optical sensor, an x-ray sensor, an RF sensor, a MW sensor, an infrared thermography sensor, or an ultrasound sensor, an MR sensor, an impedance sensor, a temperature sensor, a biosensor, a chemical sensor, a radioactive-emission sensor, and a mechanical sensor.

54. The method of claim 51, wherein the communication line further includes an instrument bundle.

55. The method of claim 51, wherein the second procedure includes taking a biopsy sample.

56. The method of claim 51, wherein the second procedure includes a localized surgery.

57. The method of claim 51, wherein the second procedure includes dispensing medication.

58. The method of claim 51, wherein the second procedure includes characterizing the tissue with an additional sensor.

59. A method for tissue characterization, comprising:
inserting a guide wire intracorporeally;
inserting intracorporeally, along the guide wire, an electromagnetic sensor for tissue characterization, operative in an electromagnetic frequency range of less than 100 Ghz, wherein the sensor is mounted on a communication line; and
characterizing the tissue with the sensor.

60. The method of claim 59, wherein the electromagnetic sensor for tissue characterization is a nonirradiative electromagnetic sensor.

61. (canceled)

62. The method of claim 59, wherein the communication line includes an instrument bundle.

63. The method of claim 59, and further including:
removing the sensor for tissue characterization after the characterizing the tissue;
inserting a second instrument, mounted on a second communication line, intracorporeally, along the guide wire.

64. The method of claim 63, wherein the second instrument is a biopsy instrument.

65. The method of claim 63, wherein the second instrument is configured for a localized surgery.

66. The method of claim 63, wherein the second instrument is configured for dispensing medication.

67. The method of claim 63, wherein the second instrument is a sensor, selected from the group consisting of an optical sensor, an X-ray sensor, an RF sensor, a MW sensor, an infrared thermography sensor, or an ultrasound sensor, an MR sensor, an impedance sensor, a temperature sensor, a biosensor, a chemical sensor, a radioactive-emission sensor, and a mechanical sensor.

68. The method of claim 63, wherein the second communication line includes an instrument bundle.

69. An endoscope system, which comprises:
an endoscope, comprising:
an intracorporeal portions, configured for insertion into a body, and including:
an electromagnetic sensor for tissue characterization, operative in an electromagnetic frequency range of less than 100 Ghz;
a communication line, on which the electromagnetic sensor is mounted; and
an extracorporeal portion, configured for manipulating the intracorporeal portion; and
a control station.

70. The system of claim 69, wherein the control station further includes at least one of a control unit, control buttons, a keyboard, a read/write device, a signal analyzer, and a display screen.

71. The system of claim 69, wherein the electromagnetic sensor is a nonirradiative electromagnetic sensor.

72. The endoscope of claim 10, wherein the second instrument is configured for taking a biopsy sample.

73. The endoscope of claim 10, wherein the second instrument is configured for localized surgery.

74. The endoscope of claim 10, wherein the second instrument is configured for dispensing medication.

75. The endoscope of claim 13, wherein the intracorporeal portion includes a cutting tool, configured to facilitate entry to the body lumen by percutaneous insertion.

76. The endoscope of claim 13, wherein the intracorporeal portion includes a cutting tool configured for penetrating the wall of the body lumen, for interacting with a tissue outside the body lumen.

77. The endoscope of claim 1, wherein the electromagnetic sensor further includes a cutting tool, thus forming an integrated sensing-cutting device.

78. The endoscope of claim 1, wherein the electromagnetic sensor is a nonirradiative electromagnetic sensor.

79. The endoscope of claim 78, wherein the nonirradiative electromagnetic sensor is configured for:
applying an electrical pulse to the tissue;
generating an electrical fringe field in a near-field zone of the tissue, so as to produce a reflected pulse from the near-field zone of the tissue with negligible radiation penetrating the tissue; and
detecting the reflected electrical pulse.

80. The endoscope of claim 78, wherein the nonirradiative electromagnetic sensor includes:
a resonating element, formed as a conductive structure, configured to be placed proximally to an edge of the tissue, without penetrating the tissue, and having a diameter-equivalent D, which defines a cross-sectional area of the resonating element, on a plane substantially parallel with the edge of the tissue; and
at least one conductive lead, for providing communication with an external system,
wherein the resonating element is configured to resonate at a free-air wavelength range of between about λ and about 10λ, wherein λ is at least about ten times the diameter-equivalent D, and wherein upon receiving a signal in the range of between about λ and about 10λ, the electromagnetic sensor is configured to induce electric and magnetic fields, in a near zone, in the tissue, the near zone being a hemisphere having a diameter of substantially D, beginning with the edge of the tissue, while causing negligible radiation in a far zone, so that the tissue, in the near zone, effectively functions as part of the resonating element,
and wherein different tissue types have different resonating responses to the electromagnetic sensor, so that the tissue, in the near zone, may be categorized, by the resonating response to the nonirradiative electromagnetic sensor.

81. The endoscope of claim 1, wherein the electromagnetic sensor is operative in an electromagnetic frequency range of less than 10 Ghz.

82. The method of claim 21, wherein the electromagnetic sensor is a nonirradiative electromagnetic sensor.

83. The method of claim 82, wherein the nonirradiative electromagnetic sensor is configured for:
applying an electrical pulse to the tissue;
generating an electrical fringe field in a near-field zone of the tissue, so as to produce a reflected pulse from the near-field zone of the tissue with negligible radiation penetrating the tissue; and
detecting the reflected electrical pulse.

84. The method of claim 82, wherein the nonirradiative electromagnetic sensor includes:
a resonating element, formed as a conductive structure, configured to be placed proximally to an edge of the tissue, without penetrating the tissue, and having a diameter-equivalent D, which defines a cross-sectional area of the resonating element, on a plane substantially parallel with the edge of the tissue; and
at least one conductive lead, for providing communication with an external system,
wherein the resonating element is configured to resonate at a free-air wavelength range of between about λ and about 10λ, wherein λ is at least about ten times the diameter-equivalent D, and wherein upon receiving a signal in the range of between about λ and about 10λ, the electromagnetic sensor is configured to induce electric and magnetic fields, in a near zone, in the tissue, the near zone being a hemisphere having a diameter of substantially D, beginning with the edge of the tissue, while causing negligible radiation in a far zone, so that the tissue, in the near zone, effectively functions as part of the resonating element,
and wherein different tissue types have different resonating responses to the electromagnetic sensor, so that the tissue, in the near zone, may be categorized, by the resonating response to the nonirradiative electromagnetic sensor.

85. The method of claim 21, wherein the electromagnetic sensor is operative in an electromagnetic frequency range of less than 10 Ghz.

86. The method of claim 24, and further including a catheter, wherein the electromagnetic sensor is inserted into the catheter, and the catheter is inserted into the instrument channel.

87. The method of claim 86, wherein the catheter extends beyond a distal-most end of the endoscope, with respect to an operator, and a distal-most end of the catheter may be manipulated independently of the distal-most end of the endoscope.

88. The method of claim 86, wherein the distal-most end of the catheter is manipulated electronically.

89. The method of claim 86, wherein the distal-most end of the catheter is manipulated manually.

90. The endoscope of claim 7, wherein the distal-most end of the catheter is manipulated electronically.

91. The endoscope of claim 7, wherein the distal-most end of the catheter is manipulated manually.
